(19) United States
(12) Patent Application Publication
(10) Pub. No.: US 2006/0271199 A1
(43) Pub. Date: Nov. 30, 2006

(54) Navigational markers in implants

(76) Inventor: Lanny L. Johnson, Okemos, MI (US)

Correspondence Address: Wilson Daniel Swayze, Jr., 3804 Clearwater Ct., Plano, TX, 75025, US

(21) Appl. No.: 11/435,619

(22) Filed: May 17, 2006

Related U.S. Application Data
(60) Provisional application No. 60/682,889, filed on May 20, 2005.

(51) Int. Cl. A61F 2/28; A61B 5/11

(52) U.S. Cl. 623/18.12; 600/431; 128/903; 623/914

(57) ABSTRACT

A prosthetic implant for the body having an associated gyroscope, accelerometer and/or magnetometer as the sensor to detect changes in position of the implant. The device incorporates a miniature gyroscope, accelerometer, and/or magnetometer as a permanent part of the prosthesis for navigational information. Alternatively, the magnetometer can be external to the implant when the implant has magnetic elements associated with it. The sensors can be microelectromechanical system (MEMS) sensors placed within a drilled hole and sealed with a screw or a cap (20). The implant can be responsive to an external interrogation and powering systems. The device provides information so as to help to properly place the prosthetic implant during surgery and to assess proper functioning. The gyroscope, accelerometer, and/or magnetometer are used after implantation to provide diagnostic information in situ based upon the changed positioning or motion of the device in the prosthesis. The device can be a total joint replacement, dental implant or other type of prosthetic implant.

### PRIORTY

The present invention claims priority under 35 USC section 119 based on a provisional patent application serial No. 60/682,889 filed on May 20, 2005.

### BACKGROUND OF THE INVENTION

(1) Field of the Invention

The present invention relates generally to prosthetic implants, and more particularly to prosthetic implants having associated sensors. Specifically, the prosthetic implants have an associated gyroscope, accelerometer and/or magnetometer as the sensor to detect changes in position of the implant.

(2) Description of the Related Art

Presently, the major initiatives in surgery are robotics and navigational assist of the procedures, including implantation of devices and artificial body parts. The application is for any field of surgery, but specifically in orthopedics for total joint replacement and artificial ligament reconstruction. Specifically in surgery, the navigational systems presently used are large, require space occupying instrumentation and are monitored by video camera capture and computer analysis. There is a need for miniature systems which take up less of the operative field than those used presently used in navigational surgery.

U.S. Pat. No. 6,447,448 to Ishikawa et al. discloses an orthopedic ball sensor capable of sensing pressure, tensile strength, strain, position, and compression forces associated with prosthetics and surgically implanted devices. The device can be remotely energized and interrogated, such that the data signals are transmitted by radio waves outside of the body for reception. Ishikawa et al. teach implanting an array of sensor balls upon an artificial hip joint implant for instance along the interface with the body to assess tensile or compressive forces to monitor for instability and proper hip joint function. This provides early warning of the need for revision arthroplasty. Ishikawa et al. does not teach the use of gyroscopes in the devices.

U.S. Patent Application Publication No. 2002/0138153 to Koniuk which discloses an auto-adjusting prosthetic ankle apparatus having a damping mechanism which adjusts according to changes in ground surfaces. The prosthetic preferably has an accelerometer as an incline sensor associated with it.

U.S. Patent Application Publication No. 2004/0093093 to Andrews discloses an accelerometer and gyroscope attached to a patient or walking aid for generating signals for feedback or feedforward to a controller in a neural prosthesis.

While the related art teach sensors associated with prosthetic devices, there still exists a need for prosthetic implants having accelerometers, magnetometers, and gyroscopes.

### OBJECTS

Therefore, it is an object of the present invention to provide prosthetic implants having an associated gyroscope, magnetometer, and accelerometer.

It is further an object of the present invention to provide methods of using these prosthetic implants.

These and other objects will become increasingly apparent by reference to the following description.

### SUMMARY OF THE INVENTION

The present invention provides a device for replacement of a damaged body part comprising: a prosthetic implant; and at least one sensor affixed to the implant comprising (1) an accelerometer to provide a sensor output in response to acceleration of the implant, (2) a gyroscope to provide a sensor output in response to positioning of the implant, or (3) a magnet to provide a sensor output to a magnetometer in response to positioning of the implant, alone or in combination, and a processor to generate a data output in response to the change in sensor output. In further embodiments the implant is an orthopedic joint replacement or a dental implant.

The present invention provides device for replacement of a damaged body part comprising: a prosthetic implant; and a circuit in the implant having a power element to provide power to the circuit, at least one sensor affixed to the implant comprising (1) an accelerometer to provide a sensor output in response to acceleration of the implant, (2) a gyroscope to provide a sensor output in response to positioning of the implant, or (3) a magnetoelectric element to provide a sensor output in response to positioning of the implant in a magnetic field, alone or in combination, and a processor to generate a data output in response to the sensor output. The natural polar magnetic field or one created in the room is used for a control environment.

In further embodiments the implant is a orthopedic joint replacement or a dental implant. In still further embodiments the circuit is an integrated circuit. Preferably the accelerometer, gyroscope or magnetoelectric are provided as microelectromechanical systems (MEMS) devices. In some embodiments the power element comprises a battery to actively power the circuit. In preferred embodiments the power element comprises an inductor and regulator for receiving electromagnetic radiation energy so as to passively power the circuit.

The present invention provides a system for the determination of initial and subsequent position, subsequent loosening, and micromotion of an implant in a patient's body comprising: prosthetic implant and a circuit in the implant having a battery to provide power to the circuit, at least one sensor affixed to the implant comprising (1) an accelerometer to provide a sensor output in response to acceleration of the implant, (2) a gyroscope to provide a sensor output in response to positioning of the implant, or (3) a magnetoelectric element to provide a sensor output in response to positioning of the implant in a magnetic field, alone or in combination, a processor to generate a data output in response to the sensor output, and a transponder to generate a signal in response to the data output when the circuit is supplied with power; and a remote receiving unit for receiving the data signal generated by the transponder outside of the patient's body to determine the position, loosening, and micromotion of the implant. In further embodiments the implant is a orthopedic joint replacement or a dental implant. In still further embodiments the circuit is an integrated circuit. Preferably the gyroscope, accelerometer, and/or magnetoelectric are provided as microelectromechanical (MEMS) and/or magnetic fields systems devices.

The present invention provides a system for the determination of any loosening and micromotion of an implant in a patient's body comprising: prosthetic implant and a circuit in the implant having a battery to provide power to the circuit, at least one sensor affixed to the implant comprising (1) an accelerometer to provide a sensor output in response to acceleration of the implant, (2) a gyroscope to provide a sensor output in response to positioning of the implant, or (3) a magnetoelectric element to provide a sensor output in response to positioning of the implant in a magnetic field, alone or in combination, a processor to generate a data output in response to the sensor output, and a transponder to generate a signal in response to the data output when the circuit is supplied with power; and a remote powering/receiving unit for supplying energy to the inductor and receiving the data signal generated by the transponder outside of the patient's body, wherein when power is supplied to the circuit by the powering/receiver unit the sensor provides a sensor output in response to acceleration or positioning of the implant, and the powering/receiver unit receives the data signal from the transponder to determine whether any loosening and micromotion of the implant is occurring. In further embodiments the implant is a orthopedic joint replacement or a dental implant. In still further embodiments the circuit is an integrated circuit. Preferably the accelerometer, gyroscope and/or the magnetoelectric are provided as microelectromechanical systems (MEMS) devices.

The present invention provides a method of determining whether any subsequent loosening and micromotion of an implant in a patient's body is occurring comprising: providing the patient with an prosthetic implant and a circuit in the implant having a battery to provide power to the circuit, at least one sensor affixed to the implant comprising (1) an accelerometer to provide a sensor output in response to acceleration of the implant, (2) a gyroscope to provide a sensor output in response to positioning of the implant, or (3) a magnetoelectric element to provide a sensor output in response to positioning of the implant in a magnetic field, alone or in combination, a processor to generate a data output in response to the sensor output, and a transponder to generate a signal in response to the data output when the circuit is supplied with power; providing a remote receiver unit for receiving the data signal generated by the transponder outside of the patient's body, wherein the receiver unit receives the data signal from the transponder to determine the movement and positioning of the implant; generating a data signal in response to the sensor output; receiving the data signal with the powering/receiving unit; and determining whether any loosening and micromotion of the implant is occurring from the data signal.

In further embodiments the implant is an orthopedic joint replacement or a dental implant. In still further embodiments the circuit is an integrated circuit. Preferably the gyroscope, accelerometer, and/or magnetoelectric are provided as microelectromechanical systems (MEMS) devices.

The present invention provides a method of determining whether any loosening and micromotion of an implant in a patient's body is occurring comprising: providing the patient with an prosthetic implant and a circuit in the implant having a inductor and regulator to provide power to the circuit, at least one sensor affixed to the implant comprising (1) an accelerometer to provide a sensor output in response to acceleration of the implant, (2) a gyroscope to provide a sensor output in response to positioning of the implant, or (3) a magnetoelectric element to provide a sensor output in response to positioning of the implant in a magnetic field, alone or in combination, a processor to generate a data output in response to the sensor output, and a transponder to generate a signal in response to the data output when the circuit is supplied with power; providing a remote powering/receiving unit for supplying energy to the inductor and receiving the data signal generated by the transponder outside of the patient's body, wherein when power is supplied to the circuit by the powering/receiver unit the sensor provides a sensor output in response to acceleration or positioning of the implant, and the powering/receiver unit receives the data signal from the transponder to determine the movement and positioning of the implant; supplying energy to the inductor to power the circuit with the powering/receiving unit so as to provide a first output in response to acceleration of the implant and a second output in response to positioning of the implant; generating a data signal in response to the first output and the second output; receiving the data signal with the powering/receiving unit; and determining whether any loosening and micromotion of an implant in a patient's body is occurring from the data signal.

In further embodiments the implant is an orthopedic joint replacement, a dental implant, or any other implant. In still further embodiments the circuit is an integrated circuit. Preferably the gyroscope, accelerometer, and/or magnetoelectric are provided as microelectromechanical systems (MEMS) devices.

### BRIEF DESCRIPTION OF THE DRAWINGS

The invention may be understood by reference to the following description taken in conjunction with the accompanying drawings, in which, like reference numerals identify like elements, and in which:

FIG. 1 is an illustration of one embodiment of a prosthetic hip implant 10 of the present invention, having an associated gyroscope and accelerometer and magnetoelectric element sealed beneath a screw cap 20.

FIG. 2 is a cross-section of the prosthetic hip implant 10 viewed along line 2-2 of FIG. 1 showing drill hole 15 in the implant 10 enclosing a circuit board 25 having an accelerometer 30, gyroscope 40 and magnetoelectric element 45.

FIG. 3 is a schematic illustrating a remote powering/receiving unit 50 supplying energy to an inductor 60 and receiving a data signal generated by a transponder 70.

FIG. 4 is an illustration of another embodiment of a prosthetic hip implant 110 of the present invention having an associated magnet sealed beneath a screw cap 120 which generates magnetic field 150 which is sensed by apparatus 160 by means of a magnetoelectric sensor 165.

FIG. 5 is an illustration of another embodiment of a cross-section, using an integrated circuit and MEMS technology.

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

The term “prosthetic implant” as used herein refers to any device for replacing, substituting, or monitoring a damaged, worn or defective part of a patient's body. The term encompasses, but is not limited to orthopedic total joint replacement implants such as knee replacements, hip replacements, and shoulder replacements. The term also refers to dental implants.

The term “magnetometer” as used herein refers to any device which can measure the direction and/or the intensity of a magnetic field in which the magnetometer is placed, whether using the earth's natural field or an artificially created field for positioning. Generally such devices are magnetoelectric. Magnetometers providing three component magnetic strength and direction measurements are included; however any magnetometer is encompassed by the term. In some embodiments of the present invention a prosthetic implant having one or more magnets affixed to the implant can be used in conjunction with an external magnetometer to determine the position of the implant. Alternatively, in other embodiments of the present invention a prosthetic implant having a magnetoelectric element as the magnetometer is affixed to the implant to provide a sensor output, such that the magnetoelectric element can be utilized to determine the positioning of the implant when it is in a magnetic field.

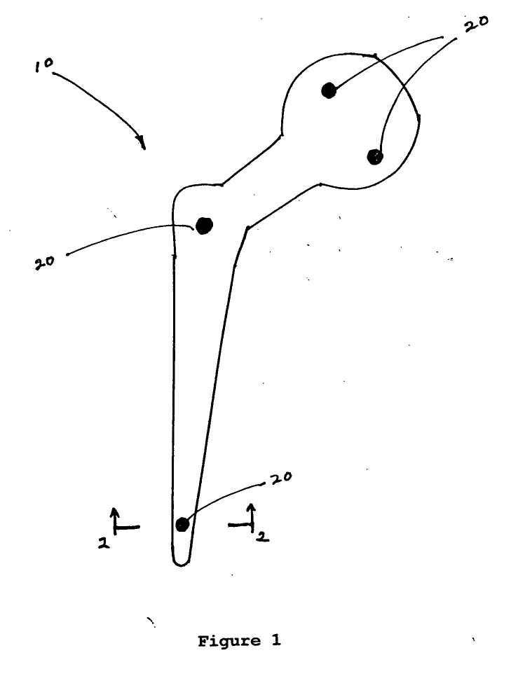

In one embodiment, the present invention provides a prosthetic implant for the body having an attached miniature gyroscope, accelerometer, and/or magnetometer used in combination or separately. Therefore, the present invention provides a device for replacement of a damaged body part comprising: (a) a prosthetic implant; and (b) a circuit in the implant having a power element to provide power to the circuit, at least one sensor affixed to the implant comprising (1) an accelerometer to provide a sensor output in response to acceleration of the implant, (2) a gyroscope to provide a sensor output in response to positioning of the implant, or (3) a magnetoelectric element to provide a sensor output in response to positioning of the implant in a magnetic field, alone or in combination, and a processor to generate a data output in response to the sensor output. An embodiment, having an accelerometer, gyroscope, and magnetoelectric element is illustrated in FIGS. 1 to 3, as described below.

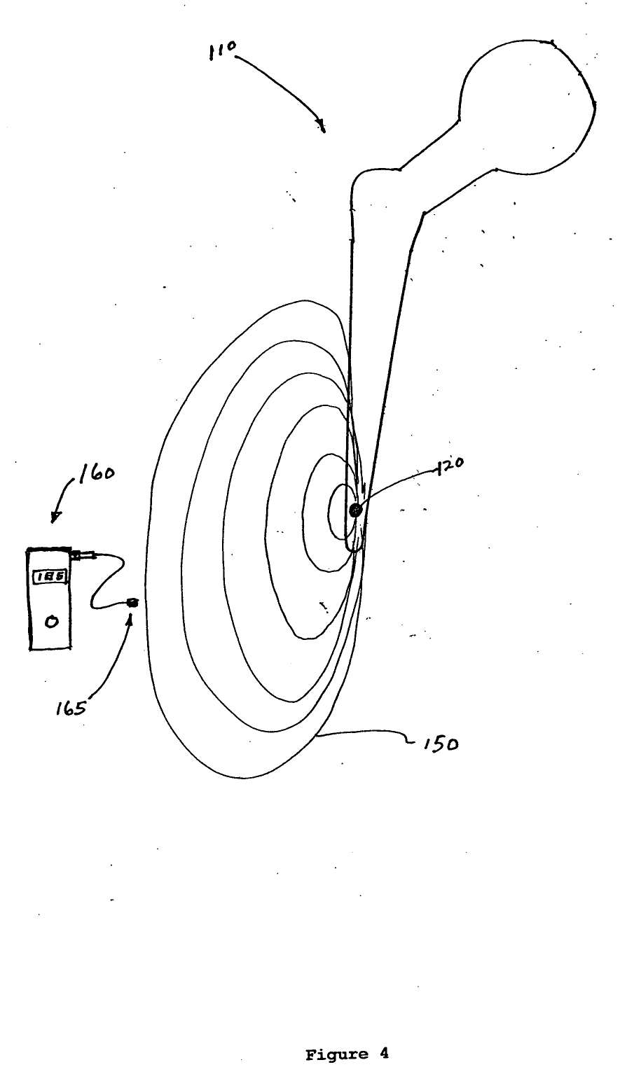

Alternatively, the present invention provides a device for replacement of a damaged body part comprising: (a) a prosthetic implant; and (b) at least one sensor affixed to the implant comprising (1) an accelerometer to provide a sensor output in response to acceleration of the implant, (2) a gyroscope to provide a sensor output in response to positioning of the implant, or (3) a magnet to provide a sensor output to a magnetometer in response to positioning of the implant, alone or in combination, and a processor to generate a data output in response to the change in sensor output. One embodiment, having a magnet affixed to the implant to provide a sensor output to a magnetometer, is illustrated in FIG. 4. The prosthetic hip implant 110 has an associated magnet attached or sealed beneath a screw cap 120 generating a magnetic field 150 which is sensed by an apparatus 160 by means of a magnetoelectric sensor 165 as the magnetometer.

Navigational devices are placed inside of surgical implants for the purpose of tracking movements in navigational surgery, especially surgery of total joint replacement. The devices in the implants can be used for tracking of any device micromotion or change in position. This would useful for facilitating surgical procedures and monitoring the outcome of surgical implant placement and subsequent micro-motion. Relating to magnetic fields, the implant material can be enough of a marker for the magnetic field or there can be localizers which serve as markers in non-magnetic implants. The implant has markers on or in it for determining the position in a magnetic field.

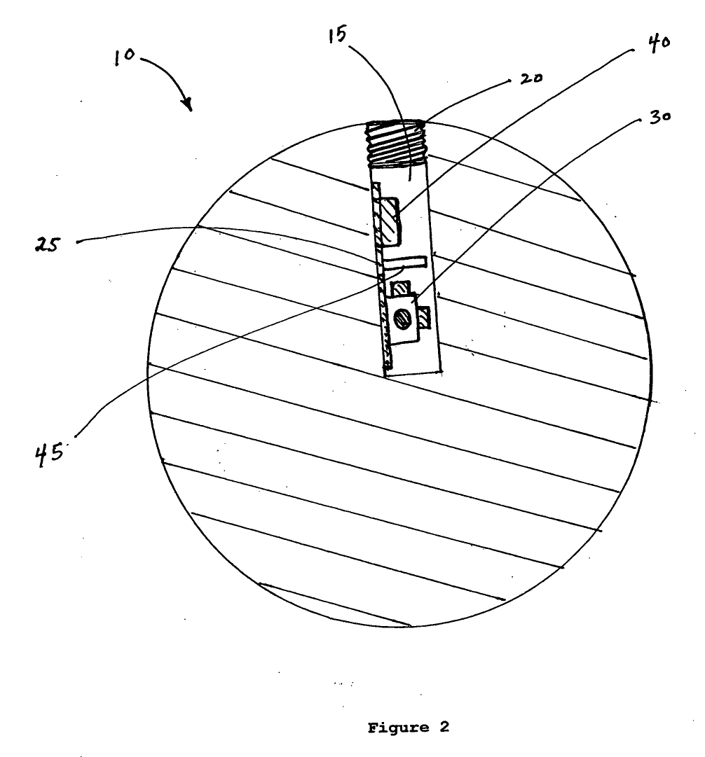

The miniature gyroscope, accelerometer, and/or magnetometer are placed inside of the total joint or other implant. In a preferred embodiment as illustrated in FIGS. 1 and 2, the gyroscope, accelerometer, and/or magnetometer can be in a drill hole 15 sealed with a screw cap 20 in the implant 10. The bore can be created after preliminary forging. The cap can be sealed by a variety of means so as not to damage the equipment. FIG. 2 shows a magnified cross-section of the prosthetic hip implant 10 viewed along line 2-2 of FIG. 1 with a drill hole 15 in the implant 10 enclosing a circuit board 25 having an accelerometer 30, gyroscope 40 and magnetoelectric element 45.

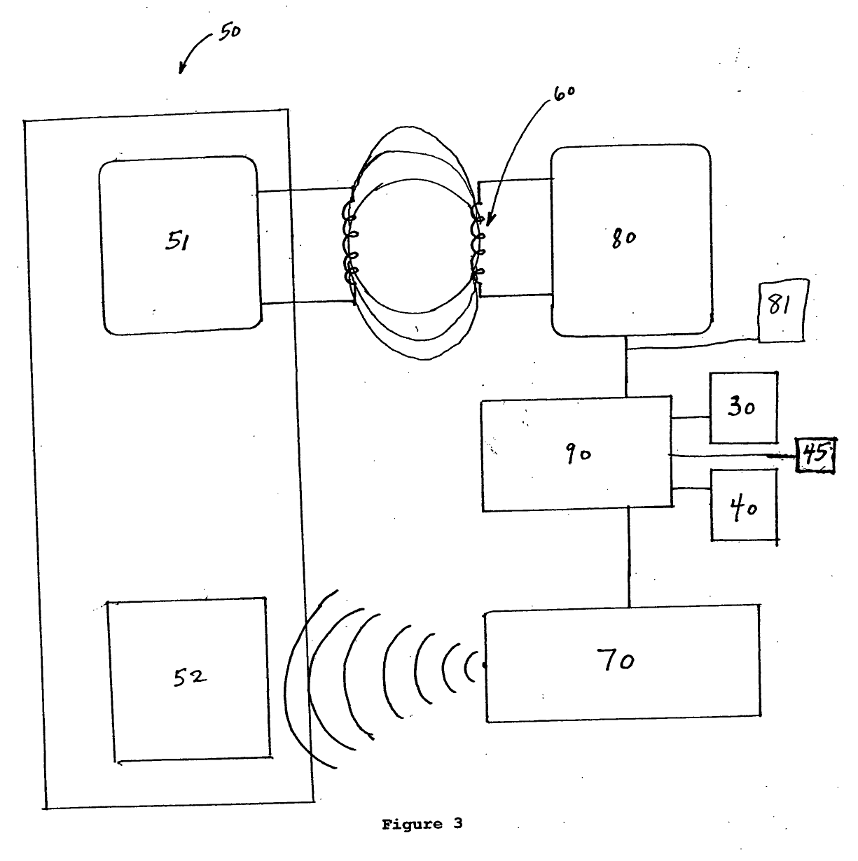

FIG. 3 is a schematic of one possible embodiment of the present invention illustrating a block circuit diagram of a remote powering/receiving unit 50 supplying energy to an inductance coil 60 and receiving a data signal generated by a transponder 70. The powering/receiving unit 50 has a power transmitter 51 to provide a magnetic field or transmit low frequency electromagnetic radiation to an inductance coil 60 so as to supply power to a power regulator 80. The power regulator 80 outputs a constant voltage to the accelerometer 30, gyroscope 40 and a processor 90. A battery 81 may receive power and store the power from the power regulator 84 from a independent source. The power from battery 81 is used to power the various attached elements. The processor 90 processes sensor output signals from the accelerometer 30, gyroscope 40 and magnetoelectric element 45. The processor 90 generates a data output in response to the sensor outputs and a transponder 70 then generates a data signal in response to the data output which is transmitted by means of one or more radiofrequencies to the receiving unit 52 of the remote powering/receiving unit 50.

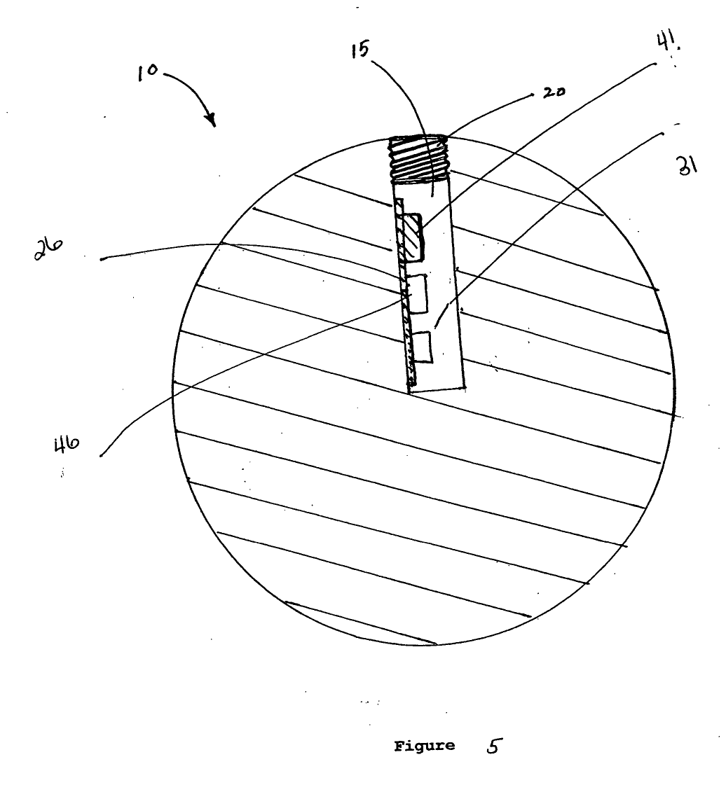

FIG. 5 shows a another magnified cross-section of the prosthetic hip implant 10 viewed along line 2-2 of FIG. 1 with a drill hole 15 in the implant 10 enclosing an integrated circuit 26 having an MEMS accelerometer 31 which has been formed by MEMS technology, MEMS gyroscope 41 which has been formed from MEMS technology and MEMS magnetoelectric element 46 which has been formed from MEMS technology. Various other configurations of this circuit apparent to one skilled in the art are encompassed by the present invention.

One benefit of the present invention is to place the implant properly. It provides immediate confirmation at surgery, when revision is still possible, if placement is not correct. In the case of bipolar implants their relationship to each other could be determined to be proper, thereby eliminating “eye balling” or “guestimates“. Postoperatively, the positioning can be compromised by micromotion and pain. There is not presently an efficient means to diagnose micromotion. The present invention makes this possible. The implants of the present invention can be used in bone to determine alignment during and after bone cutting for realignment procedures, ie. osteotomy. The present invention can be used in ligamentous reconstructive surgery for placement to determine subsequent stability and unexpected loosening. Also the present invention can be used in dental surgery implants for jaw function and occlusion versus malocclusion analysis. Any body part relationship changes can be determined. The present invention could complement other navigational circuitry or could stand-alone.

The instrumentation used in the present invention is miniature, taking up less of the operative field than that presently used in navigational surgery. More specifically, if the magnetic field method is used then little or no devices of any size are required in the operative field, thereby facilitating the surgical procedure access. In addition, when used in the course of an operation the miniature gyroscope, accelerometer, and/or magnetometer within the implant would assure correct placement of the implant. The implant could be monitored for any position changes. The most difficult position change is called micro-motion. This means that the motion is small but symptom producing, and is not detectable by present day means of imaging. Vibrations of the implant which may correspond to loosening of the implant in the body, including both translational vibration and torsional (angular) vibration, can be sensed by an implant of the present invention utilizing the gyroscope, accelerometer, and/or magnetometer individually or working together.

The technology can be used for any of a variety of body implants: dental for maxillofacial surgery, artificial ligaments, bone allografts, cosmetic surgery implants, Cochlear ear implants, neurosurgical implants, drug delivery implants. The can be robotic surgery and remote robotic surgery applications. Also, the devices can be used for training devices and surgical simulators. There are applications in gait analysis and with artificial limbs.

Propioception is the complex neurological function that allows a person to know where their body is in space. The device of the present invention can be used in artificial limbs and orthotics to monitor the position in space. There can be an adjunct device as a propioception substitute to activate electronically subsequent motion.

The device incorporates the miniature gyroscope, accelerometer, and/or magnetometer as a permanent part of the prosthesis for navigational information. Embodiments of the invention include total joint replacements, dental implants or other types of prosthetic implant for the body. In some embodiments the prosthesis is a joint replacement such as disclosed in U.S. Pat. No. 6,702,854 to Cheal et al., U.S. Pat. No. 6,319,286 to Fernanadez et al., U.S. Pat. No. 6,299,648 to Doubler et al., U.S. Pat. No. 6,264,699 to Noiles et al., U.S. Pat. No. 5,876,459 to Powell et al., U.S. Pat. No. 5,658,349 to Brooks et al., U.S. Pat. No. 5,653,765 to McTighe et al., U.S. Pat. No. 5,549,706 to McCarthy et al., and U.S. Pat. No. 5,507,830 to DeMane et al., each of which are hereby incorporated herein by reference.

Preferably the gyroscope, accelerometer, and/or magnetometer of the present invention are provided as microelectromechanical system (MEMS) sensors such as those described in U.S. Pat. No. 6,725,719 and U.S. Patent Application Publication No. 2002/0174720 to Cardarelli; U.S. Pat. Nos. 6,837,107, 6,767,758, 6,684,698, 6,505,512, 6,505,511, 6,487,908, 6,481,284, 6,122,961, 5,869,760, 5,635,640, and 5,635,638 to Geen et al.; U.S. Pat. No. 6,386,032 to Lemkin et al.; U.S. Pat. Nos. 6,192,757 and 6,009,753 to Tsang et al.; U.S. Pat. Nos. 6,148,670 and 5,939,633 to Judy; U.S. Pat. No. RE36,498 to Howe et al.; U.S. Pat. No. 5,880,369 to Samuels et al.; U.S. Pat. No. 5,847,280 to Sherman et al.; and U.S. Pat. No. 5,828,115 to Core, each of which are incorporated herein by reference in the entirety. Any commercially available MEMS sensors such as iMEMS(& accelerometers and gyroscopes (Analog Devices, Inc., Norwood, Mass.) can also be used. The sensors are placed within a hole in the prosthesis and sealed with a screw or any other type of cap to isolate it from the patient's body.

The gyroscope functions to provide information so as to help to properly place the prosthetic implant during surgery and to assess proper functioning. The gyroscope, accelerometer, and/or magnetometer are also used after implantation to provide diagnostic information in situ based upon the changed positioning, linear and angular motion of the device in the prosthesis. Methods of detecting a loosening prosthetic implants as described by Li et al., Med Eng Phys. 1996 October; 18(7):596-600, and by Chu et al., J Biomech. 1986;19(12):979-87. The implant of the present invention can be responsive to interrogation by a receiving unit external to the body to collect the data from the gyroscope, accelerometer, and/or magnetometer. Typically, the transponder and receiving unit communicate via radiofrequency signals. Transponders are known in the art and examples are disclosed in U.S. Pat. No. 4,345,253 to Hoover, incorporated herein by reference.

Preferably the circuits in the device can be powered by systems external to the body, however internal power elements such as batteries are possible. External powering systems include generators of electromagnetic radiation to provide a current in the inductor. An internal passive power element, such as an inductance coil and a regulator, is incorporated into the circuit so as to supply a constant voltage to the gyroscope, accelerometer, and/or magnetometer, and also the processor when irradiated with the electromagnetic radiation by the powering system. External interrogation and powering systems can be provided separately or as a single unitary apparatus. Some examples of interrogation and powering systems are described in U.S. Pat. No. 6,667,725 to Simons et al., U.S. Pat. No. 6,206,835 to Spillman et al., and U.S. Pat. No. 6,447,448 to Ishikawa et al., hereby incorporated herein by reference in their entirety.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the Claims attached herein.

I claim:

1) A device for replacement of a damaged body part comprising:
a prosthetic implant; and
at least one sensor affixed to the implant comprising (1) an accelerometer to provide a sensor output in response to acceleration of the implant, (2) a gyroscope to provide a sensor output in response to positioning of the implant, or (3) a magnet to provide a sensor output to a magnetometer in response to positioning of the implant, alone or in combination, and a processor to generate a data output in response to the change in sensor output.

2) The device of claim 1 wherein the implant is a orthopedic joint replacement or a dental implant.

3) A device for replacement of a damaged body part comprising:
a prosthetic implant; and
a circuit in the implant having a power element to provide power to the circuit, at least one sensor affixed to the implant comprising (1) an accelerometer to provide a sensor output in response to acceleration of the implant, (2) a gyroscope to provide a sensor output in response to positioning of the implant, or (3) a magnetoelectric element to provide a sensor output in response to positioning of the implant in a magnetic field, alone or in combination, and a processor to generate a data output in response to the sensor output.

4) The device of claim 3 wherein the implant is a orthopedic joint replacement or a dental implant.

5) The device of claim 3 wherein the circuit is an integrated circuit.

6) The device of claim 3 wherein the accelerometer, gyroscope or magnetoelectric are provided as microelectromechanical systems (MEMS) devices.

7) The device of claim 3 wherein the power element comprises a battery to actively power the circuit.

8) The device of claim 3 wherein the power element comprises an inductor and regulator for receiving electromagnetic radiation energy so as to passively power the circuit.

9) A system for the determination of position, loosening, and micromotion of an implant in a patient's body comprising:
prosthetic implant and a circuit in the implant having a battery to provide power to the circuit, at least one sensor affixed to the implant comprising (1) an accelerometer to provide a sensor output in response to acceleration of the implant, (2) a gyroscope to provide a sensor output in response to positioning of the implant, or (3) a magnetoelectric element to provide a sensor output in response to positioning of the implant in a magnetic field, alone or in combination, a processor to generate a data output in response to the sensor output, and a transponder to generate a signal in response to the data output when the circuit is supplied with power; and
a remote receiving unit for receiving the data signal generated by the transponder outside of the patient's body to determine the position, loosening, and micromotion of the implant.

10) The system of claim 9 wherein the implant is a orthopedic joint replacement or a dental implant.

11) The system of claim 9 wherein the circuit is an integrated circuit.

12) The system of claim 9 wherein the accelerometer, gyroscope or magnetoelectric are provided as microelectromechanical systems (MEMS) devices.

13) A system for the determination of any loosening and micromotion of an implant in a patient's body comprising:
prosthetic implant and a circuit in the implant having a battery to provide power to the circuit, at least one sensor affixed to the implant comprising (1) an accelerometer to provide a sensor output in response to acceleration of the implant, (2) a gyroscope to provide a sensor output in response to positioning of the implant, or (3) a magnetoelectric element to provide a sensor output in response to positioning of the implant in a magnetic field, alone or in combination, a processor to generate a data output in response to the sensor output, and a transponder to generate a signal in response to the data output when the circuit is supplied with power; and
a remote powering/receiving unit for supplying energy to the inductor and receiving the data signal generated by the transponder outside of the patient's body, wherein when power is supplied to the circuit by the powering/receiver unit the sensor provides a sensor output in response to acceleration or positioning of the implant, and the powering/receiver unit receives the data signal from the transponder to determine whether any loosening and micromotion of the implant is occurring.

14) The system of claim 13 wherein the implant is a orthopedic joint replacement or a dental implant.

15) The system of claim 13 wherein the circuit is an integrated circuit.

16) The system of claim 13 wherein the accelerometer, gyroscope or magnetoelectric are provided as microelectromechanical systems (MEMS) devices.

17) A method of determining whether any loosening and micromotion of an implant in a patient's body is occurring comprising:
providing the patient with an prosthetic implant and a circuit in the implant having a battery to provide power to the circuit, at least one sensor affixed to the implant comprising (1) an accelerometer to provide a sensor output in response to acceleration of the implant, (2) a gyroscope to provide a sensor output in response to positioning of the implant, or (3) a magnetoelectric element to provide a sensor output in response to positioning of the implant in a magnetic field, alone or in combination, a processor to generate a data output in response to the sensor output, and a transponder to generate a signal in response to the data output when the circuit is supplied with power;
providing a remote receiver unit for receiving the data signal generated by the transponder outside of the patient's body, wherein the receiver unit receives the data signal from the transponder to determine the movement and positioning of the implant;
generating a data signal in response to the sensor output;
receiving the data signal with the powering/receiving unit; and
determining whether any loosening and micromotion of the implant is occurring from the data signal.

18) The method of claim 17 wherein the implant is a orthopedic joint replacement or a dental implant.

19) The method of claim 17 wherein the circuit is an integrated circuit.

20) The method of claim 17 wherein the accelerometer, gyroscope or magnetoelectric are provided as microelectromechanical systems (MEMS) devices.

21) A method of determining whether any loosening and micromotion of an implant in a patient's body is occurring comprising:
providing the patient with an prosthetic implant and a circuit in the implant having a inductor and regulator to provide power to the circuit, at least one sensor affixed to the implant comprising (1) an accelerometer to provide a sensor output in response to acceleration of the implant, (2) a gyroscope to provide a sensor output in response to positioning of the implant, or (3) a magnetoelectric element to provide a sensor output in response to positioning of the implant in a magnetic field, alone or in combination, a processor to generate a data output in response to the sensor output, and a transponder to generate a signal in response to the data output when the circuit is supplied with power;
providing a remote powering/receiving unit for supplying energy to the inductor and receiving the data signal generated by the transponder outside of the patient's body, wherein when power is supplied to the circuit by the powering/receiver unit the sensor provides a sensor output in response to acceleration or positioning of the implant, and the powering/receiver unit receives the data signal from the transponder to determine the movement and positioning of the implant;
supplying energy to the inductor to power the circuit with the powering/receiving unit so as to provide a first output in response to acceleration of the implant and a second output in response to positioning of the implant;
generating a data signal in response to the first output and the second output;
receiving the data signal with the powering/receiving unit; and
determining whether any loosening and micromotion of an implant in a patient's body is occurring from the data signal.

22) The method of claim 21 wherein the implant is a orthopedic joint replacement or a dental implant.

23) The method of claim 21 wherein the circuit is an integrated circuit.

24) The method of claim 21 wherein the accelerometer and the gyroscope are provided as microelectromechanical systems (MEMS) devices.
